(19) United States
(12) Patent Application Publication
(10) Pub. No.: US 2013/0079625 A1
(43) Pub. Date: Mar. 28, 2013

(54) CT - MRI HYRBRID APPARATUS AND METHOD OF IMPLEMENTING THE SAME

(71) Applicant: Georges HOBEIKA, Miami, FL (US)

(72) Inventor: Georges HOBEIKA, Miami, FL (US)

(21) Appl. No.: 13/244,531

(22) Filed: Sep. 25, 2011

(51) Int. Cl. A61B 5/055; A61B 5/05; A61B 6/00

(52) U.S. Cl. 600/411; 600/425; 600/407

(57) ABSTRACT

An apparatus and method for non-invasively obtaining an image featuring information on internal human tissues, and more particularly to an apparatus and method for integrating computed tomography (CT) or computerized axial tomography (CAT) and magnetic resonance imaging (MRI) to provide high spatial-accurate resolution images which feature tumor information on human internal tissues especially as a part of a radiotherapy treatment. The apparatus includes a first section for obtaining a first set of images of a subject using a first method, a second section for obtaining a second set of images of the subject using the second method, the second method being different from the first method, and a board for carrying the subject through the first and second sections, the board being movable between the first and second sections.

### BACKGROUND OF THE INVENTION

1. Field Of The Invention

The present invention generally relates to an apparatus and method for non-invasively obtaining an image featuring information on internal human tissues, and more particularly to an apparatus and method for integrating computed tomography (CT) or computerized axial tomography (CAT) and magnetic resonance imaging (MRI) to provide high spatial-accurate resolution images which feature tumor information on human internal tissues especially as a part of a radiotherapy treatment.

2. Discussion Of The Related Art

A CT scan is an x-ray procedure that combines many x-ray images with the aid of a computer to generate cross-sectional views. In general, a CT scan costs less, takes shorter testing time, and is very good for imaging bone structures. However, CT images have a relatively low resolution in terms of soft tissue delineation and make it difficult to define with high precision disease infiltration limits, for example, extra-capsular extension in prostate cancer, or structures including glands, small organs, nerves and vessels. Nevertheless, CT images currently are the study of choice for the evaluation of bony anatomy and bilateral adrenal glands.

And, in radiotherapy, CT images form the sole basis for radiation dose computation algorithms. In clinical practice, radiation oncologists employ CT simulation, which involves three-dimensional image acquisition of the patient body or region to be treated using CT scans, as a component of radiotherapy planning More specifically, CT simulations are taken with the patient placed in the treatment position and form the basis for planning the daily radiotherapy treatment. From these images, normal structures as well as tumors are visually indentified and delineated by oncologists. A treatment plan will then be generated to give the parameters of the radiation therapy, e.g., a particular radiation dose to the tumor, while minimizing the dose to the surrounding normal structures.

However, as technology for radiotherapy for cancer or tumor advances, one of the goals is to achieve higher precision and more sophisticated treatment plan that allows radiation dosage to be very conformal to the target or tumor, while sparing surrounding normal tissues. Therefore, although CT images alone form the sole basis for radiation dose computation algorithms, the use MRI to accompany CT scans in radiotherapy has been suggested.

MRI makes use of the property of nuclear magnetic resonance to image nuclei of atoms inside a body. An MRI machine uses a magnetic field to align the magnetization of some atoms in the body, and radio frequency fields to systematically alter the alignment of this magnetization, thereby causing the nuclei to produce a rotating magnetic field detectable by the scanner. The scanner then constructs an image of the scanned area of the body. Three-dimensional images offer gradients and provide excellent contrast between the different soft issues of a body. As such, MRI currently is the ideal imaging study for delineation of tumor and normal structures. In recent years, MRI has become a companion imaging to CT scans in radiotherapy planning to assist in delineating tumor from surrounding normal structures.

Currently, CT scanner and MRI scanner are located separately from one another and a patient is transported to separate rooms to collect CT images and MRI images. Subsequently, physics personnel skills and computer software programs are used to align and fuse CT simulation axial images with MRI axial images. The fused images are then used to help in identifying and contouring a tumor target and normal structures on the CT scan images, while comparing them head to head with the MRI images of the same axial plane. However, this method of acquiring and fusing the CT and MRI images has major limited accuracy.

Oftentimes, the CT and the MRI are performed at different times and in different body positions and the images generated from both studies are not exactly at the same level or in the same orientation. It should be mentioned that even minor differences in body positioning can generate a notable discrepancy between both sets of images and impair the quality of the “fusion”. The accuracy of the MR guided contouring process can be reduced. Reduced accuracy defeats the purpose of including an MRI study as part of the simulation and treatment planning

Therefore, what is needed is a device that can reduce and minimize differences in body positioning between scans. Also needed is a device that can increase accuracy of the simulation so that treatment planning can be improved.

### SUMMARY OF THE INVENTION

Accordingly, embodiments of the invention are directed to an apparatus and method for non-invasively obtaining an image featuring information on internal human tissues that substantially obviate one or more of the problems due to limitations and disadvantages of the related art.

An object of embodiments of the invention is to provide an apparatus and method for non-invasively obtaining an image featuring information on internal human tissues that integrate at least computed tomography (CT) or computerized axial tomography (CAT) and magnetic resonance imaging (MRI).

An object of embodiments of the invention is to provide an apparatus and method for non-invasively obtaining an image featuring information on internal human tissues that provide a single scanning board between at least two different types of imaging machines.

Additional features and advantages of embodiments of the invention will be set forth in the description which follows, and in part will be apparent from the description, or may be learned by practice of embodiments of the invention. The objectives and other advantages of the embodiments of the invention will be realized and attained by the structure particularly pointed out in the written description and claims hereof as well as the appended drawings.

To achieve these and other advantages and in accordance with the purpose of embodiments of the invention, as embodied and broadly described, an apparatus and method for non-invasively obtaining an image featuring information on internal human tissues according to an embodiment of the present invention includes a first section for obtaining a first set of images of a subject using a first method, a second section for obtaining a second set of images of the subject using the second method, the second method being different from the first method, and a board for carrying the subject through the first and second sections, the board being movable between the first and second sections.

A method for non-invasively obtaining an image featuring information on internal human tissues according to an embodiment of the present invention includes providing a movable board, carrying a subject on the movable board through a first section of the procedure and a second section of the procedure, moving the board into the first section and performing a first examination method on the subject while the subject is on the board, moving the board into the second section and performing a second examination method on the subject while the subject is on the board, wherein the first and second examination methods being different from one another; and combining results from the first and second examination methods for overlapping common information about the subject.

It is to be understood that both the foregoing general description and the following detailed description are exemplary and explanatory and are intended to provide further explanation of embodiments of the invention as claimed.

### BRIEF DESCRIPTION OF THE DRAWINGS

The accompanying drawings, which are included to provide a further understanding of embodiments of the invention and are incorporated in and constitute a part of this specification, illustrate embodiments of the invention and together with the description serve to explain the principles of embodiments of the invention.

FIG. 1 is a side view of an image scanner according to an embodiment of the present invention.

### DETAILED DESCRIPTION OF THE PREFERRED EMBODIMENTS

Reference will now be made in detail to the preferred embodiments of the invention, examples of which are illustrated in the accompanying drawings.

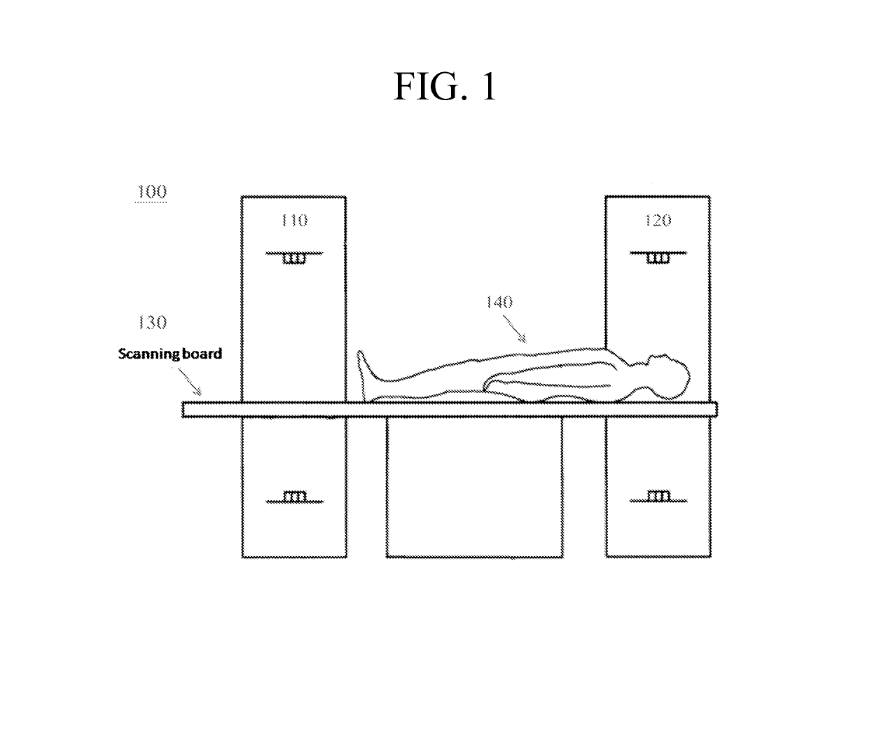

FIG. 1 is a side view of an image scanner according to an embodiment of the present invention. As shown in FIG. 1, a system 100 includes a first section 110 for obtaining images based on a first method and a second section 120 for obtaining images based on a second method. The first method may be based on computed tomography (CT) or computerized axial tomography (CAT). The second method may be based on magnetic resonance imaging (MRI).

The system 100 further includes an elongated scanning board 130. A subject 140, such as a patient, is fixed onto the board 130 using immobilization devices such as masks or cradles.

After the subject 140 is fixed onto the board 130, the subject 140 preferably first passes through the first section 110 and subsequently through the second section 120. The board 130 is movable and slidable along an axial through the first and second sections 110 and 120. While going through the first and second sections 110 and 120, the subject 140 lies still with immobilization devices and undergoes a continuous multi-step scanning process. For example, a CT scan of the area of interest of the subject 140 can be followed by an MRI scan of that same area of interest.

A feature of the system 100 and the method of obtaining images using the system 100 is that the subject 140 remains immobilized in the same exact position on the scanning board 130 during the entire image acquisition process. Thus, set-up errors or positioning uncertainties that could have been generated by separate scanning procedures are minimized or eliminated.

Additionally, the system 100 can obtain two different types of scans almost near simultaneous in time. Thus, any possible internal organ motion or change in configuration or size of certain tissues if both scans were more separated in time can be avoided. For example, as many organs, such as prostate gland, rectum, bladder, bowel, surgical bed, seromas and brain tissue, shift with time. The system 100 that obtain different types of scans immediately following one and another can provide even more accurate images for fusing or treatment planning

The system 100 performs different types of scans to generate a highly accurate super-imposed sets of images that can be fused. For example, the imposed images or fused images, such as head to head in an axial plan, can be within sub-millimeter accuracy. Highly accurate images obtained by the system 100 subsequently provide a confident and precise delineation of structures on the CT images guided by the corresponding MRI images. Further, the images obtained by the system 100 provide a more realistic time representation of the actual anatomy displayed on the CT images.

It will be apparent to those skilled in the art that various modifications and variations can be made in the chassis structure of embodiments of the invention without departing from the spirit or scope of the invention. Thus, it is intended that embodiments of the invention cover the modifications and variations of this invention provided they come within the scope of the appended claims and their equivalents.

What is claimed:

1. A device, comprising:
a first section for obtaining a first set of images of a subject using a first method;
a second section for obtaining a second set of images of the subject using the second method, the second method being different from the first method; and
a board for carrying the subject through the first and second sections, the board being movable between the first and second sections.

2. The device according to claim 1, further comprising a processor for controlling how the board is moved through the first and second sections.

3. The device according to claim 1, further comprising an axial for supporting the board, wherein the board is slidable along the axial.

4. The device according to claim 1, wherein the first and second sections are located within a same physical room or space.

5. The device according to claim 1, wherein the first and second sections are located in interconnecting rooms.

6. The device according to claim 1, wherein the first section comprises a CT machine.

7. The device according to claim 1, wherein the second section comprises a MRI machine.

8. A method for medical procedure, comprising:
providing a movable board;
carrying a subject on the movable board through a first section of the procedure and a second section of the procedure;
moving the board into the first section and performing a first examination method on the subject while the subject is on the board;
moving the board into the second section and performing a second examination method on the subject while the subject is on the board, wherein the first and second examination methods being different from one another; and
combining results from the first and second examination methods for overlapping common information about the subject.

9. The method according to claim 8, wherein the moving the board into the first section and the moving the board into the second section are controlled by using a processor.

10. The method according to claim 8, wherein the moving the board into the first section and the moving the board into the second section comprise the step of sliding the board along an axial located within the first and second sections.

11. The method according to claim 8, wherein the performing the first examination method comprises the step of collecting multiple x-ray images of the subject.

12. The method according to claim 8, wherein the performing the second examination method comprises the step of collecting multiple MRI images of the subject.
